(19) United States
(12) Patent Application Publication
(10) Pub. No.: US 2014/0309680 A1
(43) Pub. Date: Oct. 16, 2014

(54) INSERTION DEVICE, IN PARTICULAR A CATHETER, FOR INSERTING A MEDICAL HYBRID IMPLANT, AND ALSO MEDICAL HYBRID IMPLANT TO BE INSERTED BY MEANS OF AN INSERTION DEVICE

(71) Applicant: BIOTRONIK AG, Buelach (CH)

(72) Inventor: Amir Fargahi, Buelach (CH)

(73) Assignee: BIOTRONIK AG, Buelach (CH)

(21) Appl. No.: 14/243,829

(22) Filed: Apr. 2, 2014

Related U.S. Application Data
(60) Provisional application No. 61/810,719, filed on Apr. 11, 2013.

(51) Int. Cl. A61F 2/24; A61F 2/958; A61F 2/95

(52) U.S. Cl. CPC A61F 2/243; A61F 2/95; A61F 2/958; A61F 2/2433
USPC 606/191

(57) ABSTRACT

An insertion device for inserting a medical hybrid implant, wherein the hybrid implant has at least two sub-implants, of which at least one first sub-implant is externally expandable and at least one second sub-implant is self-expanding, including a first insertion element, which has at least one expansion aid for expanding the at least first externally expandable sub-implant, and a second insertion element and at least one third insertion element for releasing the at least second self-expanding sub-implant, wherein the at least first externally expandable sub-implant is expandable by means of the expansion aid and the at least second self-expanding sub-implant can be released by a relative movement between the second insertion element and the at least third insertion element, wherein the first insertion element is arranged displacably inside the at least second insertion element.

### CROSS REFERENCE TO RELATED APPLICATIONS

This application claims benefit of priority to U.S. patent application Ser. No. 61/810,719 filed Apr. 11, 2013; the contents of which are herein incorporated by reference.

### TECHNICAL FIELD

The invention relates to an insertion device, in particular a catheter, for inserting a medical hybrid implant for implantation in an animal and/or human body, and also to a hybrid implant to be inserted by means of an insertion device, and to a method for inserting the medical hybrid implant with the aid of the insertion device according to the preambles of the independent patent claims.

### BACKGROUND

In the field of medicine, implants are often used that are introduced into an animal and/or human body either permanently or at least for a relatively long period of time in order to carry out replacement functions. For example, these implants could include heart pacemakers, brain pacemakers for Parkinson's patients, cardiac implants, cochlear implants, retinal implants, dental implants, implants for joint replacement, vessel prostheses, or stents. In the field of cardiac implants, valve implants are known for example, such as aortic valve implants, which perform the function of the natural aortic valve. In this case, the valve implant is fixed immediately after implantation following expansion of the implant structure and adopts the position of the natural aortic valve.

Implants are connected to catheters before insertion into the body and have to be fastened such that they can be placed precisely and released in a defined manner by the catheter at the site of use without complication. A frequent problem here is that the implant is fixed with an incorrect position, which may lead to a failure of the implant. This often occurs for example in the case of calcification, that is to say the deposition of calcium salts, in particular calcium phosphate (hydroxyapatite), on the structures of the heart and in particular in the case of highly asymmetrically calcified aortic stenosis. In order to overcome these problems, hybrid implants for example can be used, which includes two individual implants having different tasks. It is known for example from US 2011/0166636 A1 to implant either two balloon-expandable or two self-expanding stents in stages using a catheter, wherein one of the stents performs a fastening function and the other stent includes a valve implant.

One object of the invention is to specify an insertion device, with which a hybrid implant can be released in a highly precise and selective manner.

A further object can be considered the provision of a corresponding medical hybrid implant, which can be implanted accurately and reliably at a site of implantation.

In addition, a further object can be considered that of providing a method for inserting the medical hybrid implant according to the invention with the aid of the insertion device according to the invention, in which the medical hybrid implant can be inserted and released quickly, reliably and without complication.

### SUMMARY

The object is achieved in accordance with the invention by the features in the independent claims. Favorable embodiments and advantages of the invention will emerge from the other claims and the description.

A proposed insertion device for inserting a medical hybrid implant is proposed, wherein the hybrid implant includes at least two sub-implants, of which at least one first sub-implant is externally expandable and at least one second sub-implant is self-expanding, including a first insertion element, which includes at least one expansion aid for expanding the at least first externally expandable sub-implant, and a second insertion element and at least one third insertion element for releasing the at least second self-expanding sub-implant, wherein the at least first externally expandable sub-implant is expandable by means of the expansion aid and the at least second self-expanding sub-implant can be released by a relative movement between the second insertion element and the at least third insertion element, wherein the first insertion element is arranged displacably inside the at least second insertion element.

As a result of the embodiment according to the invention, an insertion device, for example a catheter, can be provided, with which the hybrid implant can be positioned particularly quickly and reliably. A secure anchoring of the hybrid implant at the site of implantation, such as an annulus, can also be provided. In addition, a compact system can be used with such an insertion device since a radial positioning of the components of the insertion device is possible, and a length of the insertion device can thus be reduced compared to prior art systems. Furthermore, the hybrid implant also has a shorter length compared to prior art implants. It has been found that the need for an additional stabilizing auxiliary apparatus at the area of implantation, such as a heart pacemaker, can thus also be reduced or can particularly advantageously be omitted.

Furthermore, advantages of systems with externally expandable and self-expanding implants can be combined in one insertion device. This would be, for example, good adaptability of the self-expanding sub-implant to a patient anatomy at the site of implantation, such as an annulus of a native cardiac valve. A stable fastening with the aid of the externally expandable sub-implant at the site of implantation would also be possible. In conventional, exclusively self-expanding implants, much material, such as nitinol, often has to be used in order to ensure sufficient stability. Due to the use of a hybrid implant according to the invention, a metal quantity could be reduced compared to prior art implants, which is particularly patient-friendly. Furthermore, different expansion mechanisms can advantageously be applied with the two unequally expandable sub-implants, which leads to a hybrid implant that can be used in a particularly versatile manner.

The partly released hybrid implant can particularly advantageously be repositioned by means of the embodiments according to the invention. This is particularly the case since the self-expanding sub-implant has merely a low radial force, which can be overcome again with a low application of force during what is known as “resheathing” process. In addition, a functional test of the hybrid implant can be carried out and, in the event of a defect, the dysfunctional hybrid implant can be removed. A further advantage is that, with the need for post-dilation of the site of implantation and/or a feeding vessel, such as an artery or vein, an expansion means or a balloon for expansion is already located in the body. This reduces the stress for the patient and reduces the duration of the intervention. Since the second insertion element is arranged axially displaceably with respect to the at least third insertion element, the self-expanding sub-implant can remain securely axially fixed to the third insertion element in spite of the release movement of the second insertion element.

In this context, a “hybrid implant” is to be understood to mean an implant formed from at least two sub-implants, wherein the sub-implants are designed so as to be spatially and/or functionally independent of one another. The hybrid implant is preferably composed of at least two separate component parts/sub-implants. In this case, a sub-implant constitutes an independently functioning and/or usable component part (reference is made to the comments below for a general definition of the term “implant”). By contrast, “externally expandable” is to be understood here to mean “passively expandable” and/or that the sub-implant is not independently expandable or plastically deformable and/or is expandable or plastically deformable by means of an externally supplied force. The passive expansion can be produced in a constructionally simple manner by means of an expansion aid, such as a mechanical, pneumatic and/or hydraulic spreader or advantageously a balloon. Here, “self-expanding” is to be understood to mean “actively expandable” and/or that the sub-implant is expanded or expandable independently or automatically, that is to say without external aid. The phrase “arranged displacably inside the at least second insertion element” is to be understood to mean that the first insertion element is arranged in a lumen of the second insertion element and can be moved relative thereto, in particular in the axial direction. In this case, the axial direction is to be understood to mean a direction from a proximal end of the insertion device toward a distal end of the insertion device and/or vice versa.

The first externally expandable sub-implant is preferably crimped onto the expansion aid. The second self-expanding sub-implant preferably bears against a radial inner wall of the second insertion element, whereby the second insertion element acts as a clamping body for the self-expanding sub-implant. In this context, a “clamping body” is to be understood to mean a body that holds another element, in particular the sub-implant, in the insertion device in a fixed position by means of a clamping effect and/or a force-locked connection. The self-expanding sub-implant is thus held captively in the insertion device in a clamped state. In the clamped state, an interaction between an expansion force of the sub-implant and a clamping force of the second insertion element holds the sub-implant in position, whereby the implant is prevented from sliding out from the insertion device or the clamping body.

The self-expanding sub-implant can be fixed in a spatially comfortable manner if the sub-implant is fixed in a protective sleeve of the second insertion element. This protective sleeve has a greater radial diameter than the second insertion element and forms a functional part of the second insertion element, that is to say it can be moved together with the second insertion element. The protective sleeve is connected captively to the second insertion element. Here, any type of connection considered expedient by a person skilled in the art can be considered for both fastening variants, such as a force-locked connection, an interlocking connection or an integrally bonded connection, for example by means of welding, soldering, screwing, nailing or adhesive bonding. With an embodiment of two connected component parts, properties such as size, material, coating, friction etc. can be matched individually to the requirements of the component part. In principle, the protective sleeve may also be formed in one piece with the second insertion element, wherein “in one piece” is to be understood to mean that the protective sleeve and the second insertion element are formed by the same component part and/or from a cast part and/or can only be separated from one another with a loss of function of at least one of the component parts.

In accordance with an advantageous embodiment of the invention, the at least third insertion element is arranged radially between the first insertion element and the second insertion element. A further function of the insertion device can thus advantageously be provided. This is achieved for example by designing the at least third insertion element to hold the second self-expanding sub-implant in position, at least in the event of an expansion of the sub-implant. This can be achieved by means of any principle considered usable by a person skilled in the art, such as a force-locked connection and/or interlocking connection, a static friction or the like. A distal end region of the at least third insertion element may preferably be formed as an implant holder, which has at least one structure, such as a hook, an eyelet, a slit, etc., which is designed to interact with a contact structure of the sub-implant. These are formed as eyelets for example, which, in the assembled state of the sub-implant, are arranged in the insertion device at the end of the sub-implant pointing toward the proximal end of the insertion device.

In addition, the insertion device may include at least one fourth insertion element, which is arranged radially inside the first insertion element, whereby a function may again be integrated. The phrase “arranged radially inside the first insertion element” is to be understood to mean that the at least fourth insertion element is arranged in a lumen of the first insertion element. In addition, it may be arranged displacably inside the second insertion element or it may be moved relative thereto, in particular in the axial direction. The at least fourth insertion element is designed for example to receive a guide element, such as an insertion wire and/or what is known as a “guide wire”. The hybrid implant can thus be fed to the site of implantation reliably and in a constructionally simple manner. A catheter tip is preferably arranged at a distal end of the guide element.

The first insertion element may thus be understood as an outer shaft (also referred to in the following text as a balloon outer shaft) of a balloon catheter and the fourth insertion element may thus be understood as an inner shaft (also referred to in the following text as a balloon inner shaft) of a balloon catheter, and the second insertion element may thus be understood as an outer shaft (also referred to in the following text as a catheter outer shaft) of a catheter and the third insertion element may thus be understood as an inner shaft (also referred to in the following text as a catheter inner shaft) of a catheter, with which a self-expanding implant can be fed and implanted. The insertion device with at least these components constitutes a four-shaft catheter system. With consideration of a shaft casing or a stabilizing tube arranged radially farthest outwardly, the insertion device can be considered as a five-shaft catheter system.

The first insertion element is inseparably connected to the at least fourth insertion element, whereby these can be moved together captively. If the first insertion element is arranged axially displacably with respect to the at least third insertion element, the expansion aid with the externally expandable sub-implant can be positioned relative to the self-expanding sub-implant in a constructionally simple manner. The definition of axially is to be understood similarly to the definition of axial direction. An axial movement play for the expansion aid can advantageously be provided if the second insertion element is arranged with respect to the at least first insertion element. A relative position of the insertion device at the site of implantation also remains uninfluenced by the release movement of the second insertion element if the second insertion element is arranged axially displacably with respect to the at least fourth insertion element. In addition, a relative position of the insertion device at the site of implantation, probed by the guide element with the catheter tip, can be securely held.

The movements can be conveyed in a constructionally simple manner if the first insertion element and/or the second insertion element and/or the third insertion element and/or the at least fourth insertion element each has at least one grip segment. In this case, the grip segment of the first and of the fourth insertion element is in each case designed, in at least one operating state, to move the first and the at least fourth insertion element independently of the second and the at least third insertion element. Since these grip segments are inseparably interconnected, both insertion elements can be operated with both grip segments. Here, the operating state may preferably constitute the positioning of the expansion aid. Furthermore, the grip segment of the second insertion element is designed, in at least one operating state, to move the second insertion element independently of each of the first, the at least third, and the at least fourth insertion element. Here, the operating state in particular represents the release of the second self-expanding sub-implant. If a movement of the insertion elements relative to one another is undesired, the respectively relevant insertion elements and/or grip segments thereof can be separably interconnected.

Each grip segment is preferably arranged at the proximal end of the insertion device or on a part of each insertion element located outside the body during the implantation process. The grip segment may be formed by a housing portion of an insertion element and/or a separate component part arranged or integrally formed on the insertion element. Good handling properties and a constructionally favorable design are provided if the grip segment of the insertion element arranged radially farthest inwardly (balloon inner shaft) is arranged in the direction of the proximal end and/or at the proximal end of the insertion device and the grip segment of the insertion element arranged radially farthest outwardly (catheter outer shaft) is arranged in the direction of the distal end of the insertion device. The two other grip segments are each arranged axially therebetween, the grip segment of the balloon outer shaft more in the direction of the proximal end, and the grip segment of the catheter inner shaft more in the direction of the distal end.

In an advantageous embodiment of the invention, the at least third insertion element has a stop, which limits a relative movement of the second insertion element with respect to the at least third insertion element. An extent of the movement of the second insertion element and therefore the release of the self-expanding sub-implant can thus be monitored and/or limited in a constructionally simple manner and intuitively for an operator. As a result, the self-expanding sub-implant can thus be released in stages in a simple manner. Here, the stop can be formed by any means considered practicable by a person skilled in the art, such as a (colored) marking, a groove, a limit stop, etc.

In order to enable a gentle, low-friction and trouble-free movement of the second insertion element during the release of the hybrid implant and also to enable a release of the hybrid implant without complication, the insertion device additionally has a shaft casing or a stabilizing tube, which extends in the circumferential direction around the insertion element arranged radially farthest outwardly and/or the second insertion element. For improved handling of the shaft casing or of the stabilizing tube and therefore of the insertion device as a whole, the shaft casing or the stabilizing tube has a grip segment in the direction of its proximal end. This grip segment is arranged further distally than the grip segment of the insertion elements arranged farthest distally, that is to say of the catheter outer shaft. The stabilizing tube and/or grip segment thereof must be fixed in its/their position(s) during implantation of the hybrid implant and in particular during the movement or withdrawal of the second insertion element.

The insertion device according to the invention can be used comfortably if, in the assembled state of the hybrid implant, the at least first externally expandable sub-implant faces the distal end of the insertion device and/or the at least second self-expanding sub-implant faces away from the distal end of the insertion device. In other words, the externally expandable sub-implant is arranged distally from the self-expanding sub-implant. The externally expandable sub-implant can thus enter an interaction region of the self-expanding sub-implant during the relative movement, in particular in the direction of the proximal end of the insertion device, of the first insertion element relative to the second insertion element (see below). In this case, the assembled state constitutes the state in which both sub-implants are fastened to/in the insertion device and the insertion device is ready for use for implantation of the hybrid implant. The externally expandable sub-implant has advantageously been assembled or crimped onto the expansion aid during fabrication of the insertion device. By contrast, the self-expanding sub-implant is preferably only loaded into the insertion device in the preparation laboratory. It can thus be ensured that the valve insert is assembled as closely as possible to the moment of implantation.

For entry of the first externally expandable sub-implant into the interaction region of the self-expanding sub-implant, the first externally expandable sub-implant also has at least one interaction region for interaction with the at least second self-expanding sub-implant, whereby the two sub-implants can interact in a particularly reliable manner.

In accordance with an advantageous embodiment, the at least second self-expanding sub-implant includes at least one valve insert, whereby the hybrid implant can perform a replacement function, in particular as a check valve, in the body. A hybrid implant matched particularly well to its replacement function can be provided if the at least one interaction region of the first externally expandable sub-implant can be arranged distally from a valve plane of the valve insert as a result of a relative movement between the first insertion element and the second insertion element of the insertion device. The hybrid implant can thus be fastened in a stable manner at the site of implantation, without damaging the valve insert and/or impairing the function thereof.

In accordance with a further aspect of the invention, a medical hybrid implant, in particular to be inserted by means of an above-described insertion device, is proposed. The medical hybrid implant, as described above, includes at least two sub-implants, of which at least one first sub-implant is externally expandable and at least one second sub-implant is self-expanding, wherein at least one interaction region of the first externally expandable sub-implant has an outer diameter in the expanded state and at least one interaction region of the at least second self-expanding sub-implant has an inner diameter in the expanded state, wherein a dimension of the outer diameter of the at least one interaction region of the first externally expandable sub-implant is adapted to a dimension of the inner diameter of the at least one interaction region of the at least second self-expanding sub-implant so that, in the event of a relative movement between a first insertion element and a second insertion element of the insertion device, at least the one interaction region of the first externally expandable sub-implant can enter the at least one interaction region of the at least second self-expanding sub-implant and the interaction region of the second self-expanding sub-implant can be fastened in the expanded state at a destination site by means of the interaction region of the first externally expandable sub-implant.

As a result of the embodiment according to the invention, a medical hybrid implant can be provided that can be positioned particularly quickly and reliably and also well anchored at the site of implantation. Furthermore, it can advantageously be adapted to the parameters or to anatomical conditions of the site of implantation, such as calcification of a blood vessel wall and/or of an annulus, and/or another congenital and/or morbid anomaly of the site of implantation. Furthermore, the hybrid implant has a shorter length compared to prior art implants. In addition, small French sizes, for example between 9 F and 15 F, can be implemented. Furthermore, with the two unequally expandable sub-implants, different expansion mechanisms can advantageously be applied, which leads to a hybrid implant that can be used in a particularly versatile manner. Advantages of both expansion mechanisms can thus be combined in one implant. This would be, for example, a good adaptability of the self-expanding sub-implant to a patient anatomy at the site of implantation, such as an annulus of a native cardiac valve. A stable fastening with the aid of the externally expandable sub-implant at the site of location would also be possible. Due to the use of such a hybrid implant, a metal quantity could be reduced compared to prior art implants, which is particularly patient-friendly. In addition, the partly released hybrid implant can be repositioned on account of a resheathing process due to the low radial force of the self-expanding sub-implant. Furthermore, a functional test of the hybrid implant can be carried out and, in the event of a defect, the dysfunctional hybrid implant can be removed.

In this context, an “implant” is to be understood to mean a body that performs at least one replacement function permanently or for a relatively long period of time upon implantation in an animal and/or human body. All medical implants appearing appropriate to a person skilled in the art would be conceivable here, such as a cardiac implant, a cochlear implant, a vessel prosthesis or an appendix prosthesis, or a design of the medical hybrid implant as a valve implant is particularly advantageously proposed. In particular, a “valve implant” is to be understood in particular to mean a body that performs at least one replacement function of a check valve, either permanently or for a relatively long period of time upon implantation. Any medical valve implants appearing appropriate to a person skilled in the art would be conceivable here, such as an aortic valve implant, a pulmonary valve implant, a mitral valve implant or a tricuspid valve implant.

A design of the medical hybrid implant as a stent, in particular a coronary stent, with an implant structure and/or valve insert connected reversibly or irreversibly to the stent is particularly advantageously proposed. In this context a “valve insert” is understood in particular to mean an aortic valve, a pulmonary valve, a mitral valve, a tricuspid valve or a venous valve made of natural and/or artificial material. An embodiment of the medical hybrid implant as an aortic valve is particularly preferable, whereby a filed replacement structure can be provided for the cardiac valve affected most frequently by malfunctions. Complications, such as faults of the mitral valve or the need for a heart pacemaker, can also conveniently be reduced. An embodiment as a pulmonary valve or an embodiment as a mitral valve is also conceivable. Generally, any other implant structure appearing expedient to a person skilled in the art would be conceivable however. A structure that can be implanted in a constructionally simple manner can be provided by the design of the hybrid implant as a stent.

It may also be advantageous if the first externally expandable sub-implant includes at least cobalt and/or chromium, preferably in the form of stainless steel or medical stainless steel and/or a Cr—Ni—Fe steel (here, preferably the alloy 316L) or a Co—Cr steel. In addition, the second self-expanding sub-implant is preferably manufactured from an elastic or superelastic material, for example a metal material and/or from a combination of a plurality of metal materials, such as iron, magnesium, nickel, tungsten, titanium, zirconium, niobium, tantalum, zinc, silicon, lithium, sodium, potassium, calcium, manganese and/or any other material appearing expedient to a person skilled in the art. A zinc/calcium alloy would also be possible. The second self-expanding sub-implant is advantageously manufactured from a shape-memory material, such as a copper/zinc/aluminum alloy and/or nickel/titanium alloy, preferably nitinol.

Furthermore, it may be advantageous if the medical hybrid implant has a separation means, which separates the material of the first externally expandable sub-implant from the material of the second self-expanding sub-implant or the interaction regions thereof. In this context, a “separation means” is to be understood in particular to mean any means appearing convenient to a person skilled in the art, such as a spacer and/or in particular a coating. In this case, a functional transfer, in particular from the externally expandable sub-implant to the self-expanding sub-implant, must be ensured however. A “coating” is to be understood to mean an at least partial and preferably complete covering of the interaction regions or braces or stent struts thereof. The coating is particularly advantageously formed by an amorphous silicon carbide. However, any other coating appearing usable to a person skilled in the art and that effectively prevents contact of the materials of the two sub-implants and in particular of the metal materials, in particular such as NiTi or CoCr, in the presence of electrolytes would generally be conceivable. A problem of contact between the different noble metals of the two regions can thus be solved by the coating in a constructionally simple and space-saving manner.

As described above, the at least second self-expanding sub-implant includes at least one valve insert, whereby the hybrid implant can perform a replacement function, in particular as a check valve, in the body. Since the self-expanding sub-implant can adapt flexibly to the patient anatomy, the valve insert can thus also advantageously adapt to the conditions.

In this case, the valve insert can be connected to the self-expanding sub-implant by means of any connection type considered appropriate by a person skilled in the art, such as sewing and/or adhesive bonding. As mentioned above, the valve material may be of artificial or natural origin (cow, pig, horse). The valve of the valve insert is preferably manufactured from bovine or porcine pericardium.

A hybrid implant matched particularly well to its replacement function can be provided if the at least one interaction region of the first externally expandable sub-implant can be arranged distally from the valve plane of the valve insert as a result of the relative movement between the first insertion element and the second insertion element of the insertion device. The hybrid implant can thus be fastened in a stable manner at the site of implantation, without damaging the valve insert and/or impairing the function thereof. In accordance with a further embodiment of the invention, the externally expandable sub-implant is therefore arranged axially before the annulus in the direction of flow of the flow medium in the intended end state. It is thus also possible to prevent the coronary artery of the heart from being covered by the externally expandable sub-implant in the aortic bulb. As a result of this embodiment of the arrangement according to the invention, the hybrid implant is adapted particularly well to the anatomy of the heart or a cardiac valve region. Here, a “direction of flow of a flow medium” is to be understood in particular to mean the scientifically known direction of flow of arterial and/or venous blood in the heart and particularly advantageously, in the case of the aortic valve, the flow of blood from the left ventricle into the aorta. In this case, the annulus is preferably the aortic annulus.

In accordance with a further advantageous embodiment, the at least second self-expanding sub-implant has a number of cells. These cells may have any shape appearing convenient to a person skilled in the art, such as a round, oval, triangular, rectangular and/or diamond shape. This shape is designed in particular to be collapsible. The cells are particularly preferably substantially diamond-shaped. The phrase “substantially diamond-shaped” is to be understood here to mean that shapes similar to a diamond or a rhombus, such as a diamond-like shape with rounded corners and/or concave and/or convex sides, are also to be understood by the term “diamond-shaped”. The cells constitute the main framework of the sub-implant. Furthermore, in this context a “main framework” is to be understood in particular to mean a structure, such as a wire braid, that substantially provides a form and/or a shape of the sub-implant. The number of cells is conveniently selected such that the second self-expanding sub-implant has a low radial force, whereby a resheathing process is possible with minimal application of force. In addition, a small crimped diameter of the second self-expanding sub-implant is thus given. Installation space can thus be saved and a patient-friendly insertion device with low French sizes in particular can be used.

The at least first externally expandable sub-implant advantageously includes at least one seal structure, which is designed, in the implanted state, to reduce a leak. In addition, the seal structure may promote an ingrowth of the hybrid implant. The seal structure may be formed for example by what is known as a “textile skirt”, which is arranged on a part of the externally expandable sub-implant and in particular on a part that, in the implanted state of the hybrid implant, faces the valve insert and/or the interaction region of the externally expandable sub-implant. The seal structure can be manufactured for example from a polyester, such as DACRON. The leak may be a valvular leak and/or a paravalvular leak for example.

Both sub-implants may have at least one anchoring means, whereby the hybrid implant can additionally be fastened at the site of implantation. In this context, an “anchoring means” is to be understood in particular to mean a loop, a hook, a tip and/or another means considered practicable by a person skilled in the art. The anchoring means can be movable independently of the two sub-implants, wherein “movable” is to be understood here in particular to mean radially movable and particularly advantageously radially movable in the direction of a wall of a blood vessel, such as an aortic wall, during the self-expansion and/or expansion by means of the expansion aid. In other words, if the shape of the site of implantation differs from an expected contour, this can be counterbalanced by the independently movable anchoring means. A variable outer contour of the hybrid implant in the implanted state can thus advantageously be set by an anchoring means. In addition, good anchoring with advantageously low application of force can be provided by the self-expansion or the expansion with the expansion aid.

A deposit-inhibiting, in particular calcification-inhibiting, coating may advantageously be provided on the implant, in particular homocysteine acid. The risk of a fault or a functional failure of the hybrid implant can thus be further reduced.

In addition, a method for inserting a medical hybrid implant with the aid of an insertion device is proposed. The method includes at least the following steps: partially expanding a first sub-region of an at least second self-expanding sub-implant, in particular of a distal end region of the at least second self-expanding sub-implant when the at least second self-expanding sub-implant is assembled on the insertion device; expanding a complete at least first externally expandable sub-implant; partially expanding a second sub-region of the at least second self-expanding sub-implant, in particular of a proximal end region of the at least second self-expanding sub-implant when the at least second self-expanding sub-implant is assembled on the insertion device.

As a result of the method according to the invention, the hybrid implant can be positioned particularly quickly and reliably, anchored and implanted. Furthermore, different expansion mechanisms can advantageously be applied with the two unequally expandable sub-implants, which leads to a method that can be used in a particularly versatile manner. Advantages of both expansion mechanisms can thus be combined in one method. A hybrid implant connected to the insertion device and already partly released can particularly advantageously be repositioned in a user-friendly manner by means of the method according to the invention. In addition, a functional test can be carried out on a hybrid implant thus fastened and the hybrid implant can be removed in the event of a defect. A further advantage is that, if there is a need for post-dilation of the site of implantation and/or a feeding vessel, such as an artery or vein, this can be carried out easily, since the expansion means or the balloon for expansion is already located in the body. The method according to the invention is a transcatheter aortic valve implantation method (TAVI method or transcatheter aortic valve replacement (TAVR) method) for a hybrid bioprosthesis.

The partial expansion of the first sub-region of the at least second self-expanding sub-implant is preferably triggered by a relative movement of the second insertion element with respect to the third insertion element of the insertion device, wherein the relative movement is limited on account of a stop of the at least third insertion element. An extent of the movement of the second insertion element and therefore the release of the self-expanding sub-implant can thus be monitored and/or restricted in a constructionally simple manner and intuitively for an operator. As a result, the self-expanding sub-implant can thus be released easily in stages. Here, the second insertion element moves in particular in the direction of the proximal end of the insertion device.

The expansion aid with the first externally expandable sub-implant is then positioned partly in the interaction region of the second self-expanding sub-implant in a positioning step, so that the first externally expandable sub-implant is arranged distally from a valve plane of the second self-expanding sub-implant. This occurs on account of a relative movement of the first insertion element with respect to the second insertion element of the insertion device, wherein the first insertion element moves in particular in the direction of the proximal end of the insertion device.

At this point or even before the positioning of the expansion aid, a positional check and/or a functional check of the valve can be carried out. If an incorrect position of the first sub-region is established or if the valve functions unsatisfactorily, a repositioning step may then be carried out. In this case, a resheathing process is carried out first. Here, the at least second insertion element is again slid over the first sub-region of the second self-expanding sub-implant by means of a relative movement with respect to the first insertion element of the insertion device, whereby the sub-implant is pushed back into its starting position. The second insertion element moves in particular in the direction of the distal end of the insertion device. The first sub-region can then be positioned by again withdrawing the second insertion element.

The first externally expandable sub-implant is advantageously expanded with the aid of at least one expansion aid, such as a balloon, whereby the first sub-region of the second self-expanding sub-implant is fastened at a site of implantation by means of an interaction region of the first externally expandable sub-implant. The first sub-region of the second self-expanding sub-implant is also the interaction region of the sub-implant. The first sub-region of the second self-expanding sub-implant can thus be fixed reliably at a site of destination, such as the site of implantation.

The partial expansion of the second sub-region of the at least second self-expanding sub-implant is favorably triggered by a relative movement of the second insertion element with respect to the third insertion element of the insertion device, whereby the hybrid implant is completely released. The hybrid implant is therefore fully implanted on the basis of a quick and low-risk method. The second insertion element moves in particular in the direction of the proximal end of the insertion device.

### DESCRIPTION OF THE DRAWINGS

The invention is explained in greater detail hereinafter by way of example on the basis of an exemplary embodiment illustrated in the drawings, in which:

FIG. 1 shows a schematic illustration of a section through a favorable exemplary embodiment of an insertion device according to the invention with an assembled hybrid implant;

FIG. 2 shows a schematic illustration of a detail of a first externally expandable sub-implant of the hybrid implant in FIG. 1;

FIG. 3 shows a schematic illustration of the first externally expandable sub-implant in FIG. 2 with a schematically drawn seal structure;

FIG. 4 shows a schematic illustration of a detail of a second self-expanding sub-implant of the hybrid implant in FIG. 1;

FIG. 5 shows a schematic illustration of the insertion device from FIG. 1 with a hybrid implant placed at the start of implantation of the hybrid implant;

FIG. 6 shows a schematic illustration of the insertion device from FIG. 1 with the second self-expanding sub-implant in FIG. 4 partly released from a second insertion element;

FIG. 7 shows a schematic illustration of the insertion device from FIG. 1 with a positioned expansion aid for expanding the first externally expandable sub-implant in FIG. 2;

FIG. 8 shows a schematic illustration of the insertion device from FIG. 1 after expansion of the first externally expandable sub-implant in FIG. 2 with the aid of the expansion aid from FIG. 7;

FIG. 9 shows a schematic illustration of the insertion device from FIG. 1 after emptying of the expansion aid;

FIG. 10 shows a schematic illustration of the insertion device from FIG. 1 with the second self-expanding sub-implant in FIG. 4 completely released from the second insertion element;

FIG. 11 shows a schematic illustration of a first part of the method steps of an insertion method according to the invention on the basis of a block diagram, and;

FIG. 12 shows a schematic illustration of a second part of the method steps of the insertion method according to the invention on the basis of a block diagram.

### DETAILED DESCRIPTION

In the figures, functionally like or similarly acting elements are denoted in each case by like reference signs. The figures are schematic illustrations of the invention. They do not show specific parameters of the invention. The figures also merely reproduce typical embodiments of the invention and are not intended to limit the invention to the embodiments illustrated. In the following text, all distal ends of the insertion device 10 are denoted by the reference sign 34, all distal ends of the insertion elements 12, 14, 16, 18 and of the shaft region 36 are denoted by the reference sign 42, and all proximal ends of the component parts of the insertion device 12, 14, 16, 18, 26, 40 are denoted by the reference sign 54, wherein the number attached by a hyphen to the respective reference sign 34, 42, 54 denotes the component part on which the corresponding end is located.

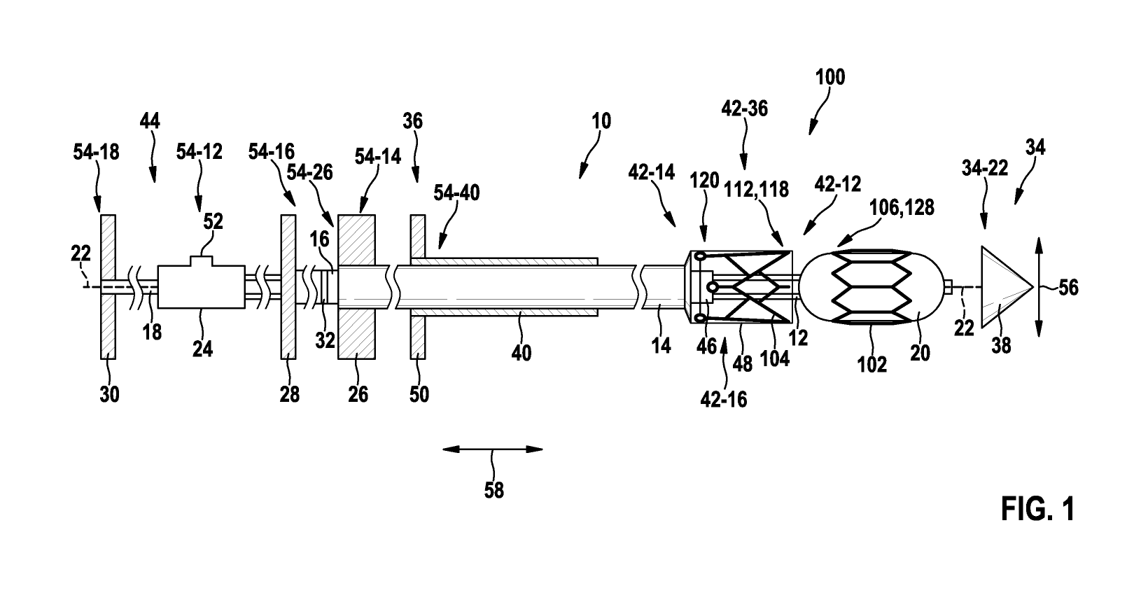

FIG. 1 shows a longitudinal section through a favorable exemplary embodiment of an insertion device 10. The insertion device 10 is used for inserting a medical hybrid implant 100 having two sub-implants 102, 104 and is a catheter for example having a shaft region 36 with four coaxially arranged insertion elements 12, 14, 16, 18, for example a balloon inner shaft (fourth insertion element 18), a balloon outer shaft (first insertion element 12), a catheter inner shaft (third insertion element 16), and a catheter outer shaft (second insertion element 14). Of these four insertion elements 12, 14, 16, 18, the second insertion element 14 is arranged radially farthest outwardly and thus surrounds the other three insertion elements 12, 16, 18.

The third insertion element 16 and the first insertion element 12 then follow inwardly in the radial direction 56, and the fourth insertion element 18 is located farthest inwardly. The third insertion element 16 is thus arranged radially between the first insertion element 12 and the second insertion element 14, and the fourth insertion element 18 is arranged radially inside the first insertion element 12. In addition, a guide element 22 is received in the fourth insertion element 18. This guide element 22 is what is known as a “guide wire”, at the distal end 34-22 of which a catheter tip 38 is arranged. The guide element 22 is indicated schematically as a dashed line in parts in FIG. 1. The second insertion element 14 arranged farthest outwardly is in turn surrounded by a shaft casing or a stabilizing tube 40. Furthermore, the first insertion element 12 includes an expansion aid 20, in the form of a balloon, for expanding the first externally expandable sub-implant 102, wherein the expansion aid 20 is arranged at a distal end 42-12 of the first insertion element 12. The second insertion element 14 is used to release the second self-expanding sub-implant 104. This occurs as a result of a relative movement in the axial direction 58 between the second insertion element 14 and the third insertion element 16. The four insertion elements 12, 14, 16, 18 and the stabilizing tube 40 have the dimensions shown in Table 1:

TABLE 1

| Insertion element (12, 14, 16, 18) | ID/OD* mm |
|---|---|
| balloon inner shaft lumen | 0.9/1.1 |
| balloon outer shaft | 1.3/1.5 |
| catheter inner shaft | 1.7/2.0 |
| catheter outer shaft | 2.2/3.0 (9 F) |
| stabilizing tube 40 | 3.2/4.0 (12 F) |

*ID = inner diameter, OD = outer diameter

As a person skilled in the art will know, these dimensions are much smaller compared to prior art devices.

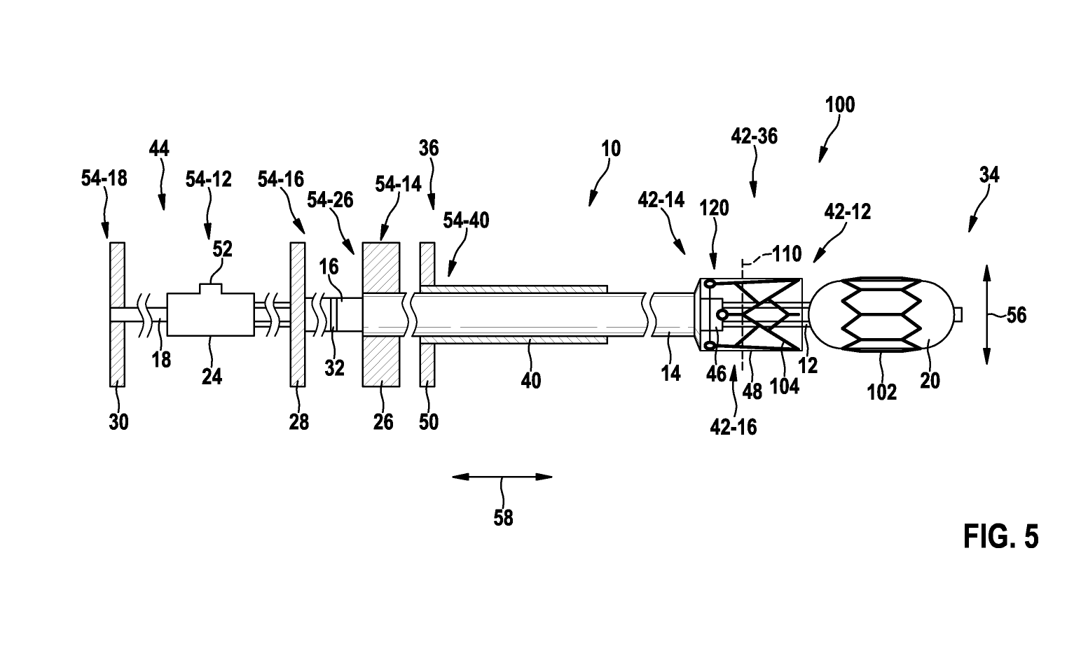

During operation, that is to say during a fastening of the hybrid implant 100 or during the implantation, the proximal end 44 of the insertion device 10 faces a user. The hybrid implant 100 is placed between the balloon inner shaft and the catheter outer shaft at a distal end 42-36 of the shaft region 36 facing away from the user, for example in the vicinity of the catheter tip 38, and is to be released at the site of implantation in the animal or human body (see FIGS. 5 to 12).

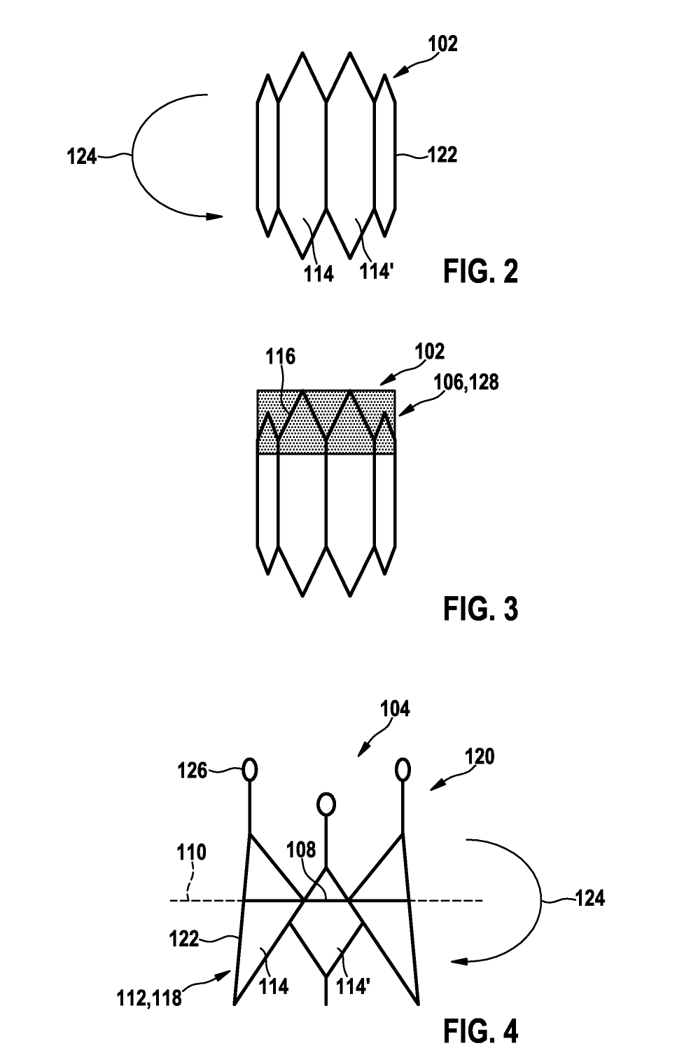

As can be seen in FIG. 1, the hybrid implant 100 includes two sub-implants 102, 104, which are each shown individually in FIGS. 2 and 4. The first sub-implant 102 is externally expandable and will be referred to in the following text as the externally expandable sub-implant 102 (see FIG. 2). The externally expandable sub-implant 102 has a homogeneous main framework 122 with a multiplicity of cells 114, 114′. The cells 114, 114′ are formed uniformly in a diamond shape and extend side-by-side in the circumferential direction 124 over a circumference of the externally expandable sub-implant 102. In addition, the externally expandable sub-implant 102 is manufactured for example from medical stainless steel or a Co—Cr alloy. During manufacture of the insertion device 10, the externally expandable sub-implant 102 is crimped onto the expansion aid 20 of the insertion device 10. In this case, a French size of 15 F or even of 12 F may be reached.

As is shown in FIG. 3, the externally expandable sub-implant 102 has a seal structure 116. This is designed, in the implanted state, to reduce a leak, in particular a valvular and/or a paravalvular leak. The seal structure 116 additionally promotes an ingrowth of the hybrid implant 100 at the site of implantation, such as an aortic annulus (not shown). In the implanted state of the externally expandable sub-implant 102, the sub-implant is arranged such that the seal structure 116 points toward a valve insert 108 of the second self-expanding sub-implant 104 (see below). The seal structure 116 may be formed for example by what is known as a “textile skirt” and for example is manufactured from a polyester, such as DACRON.

The second sub-implant 104 is self-expanding and will be referred to in the following text as the self-expanding sub-implant 104 (see FIG. 4). The self-expanding sub-implant 104 is manufactured from a shape-memory material, such as nitinol. In addition, the self-expanding sub-implant 104 has a main framework 122 with a number of cells 114, 114′, which is selected such that the second self-expanding sub-implant 104 has a low radial force, as a result of which it can be compressed again with a low application of force as required. The cells 114, 114′ are substantially diamond-shaped and are arranged side-by-side in the circumferential direction 124, offset in height in an alternating manner. Furthermore, the second self-expanding sub-implant 104 includes the valve insert 108, for example in the form of an artificial aortic valve made of porcine pericardium.

Three fastening eyelets 126 are arranged and/or integrally formed in this embodiment on an end region 120 of the self-expanding sub-implant 104, which, when assembled on the insertion device 10, points toward the proximal end 44 thereof. During preparation of the insertion device 10 in the catheter laboratory, the self-expanding sub-implant 104 is fastened to an implant holder 46, which is located at a distal end 42-16 of the third insertion element 16. For this purpose, the implant holder 46 for example includes hooks (not shown in greater detail), in which the fastening eyelets 126 of the self-expanding sub-implant 104 engage (not shown in detail). The third insertion element 16 is thus designed, in the event of an expansion of the second self-expanding sub-implant 104, to hold the sub-implant in position.

Once the self-expanding sub-implant 104 has been fastened to the implant holder 46, the self-expanding sub-implant 104 is covered by a protective sleeve 48. This protective sleeve 48 is arranged or integrally formed on a distal end 42-14 of the second insertion element 14. For this purpose, the protective sleeve 48 for example has an inner diameter (ID) of approximately 4.2 mm and an outer diameter (OD) of approximately 5.0 mm and therefore a French size of 15 F. These dimensions are generally determined in accordance with the hybrid implant 100 or valve insert 108 used. The implant is covered by moving the second insertion element 14 in the direction of the distal end 34 of the insertion device 10.

Both sub-implants 102, 104 have interaction regions 106, 112, which are designed so that the two sub-implants 102, 104 can interact with one another. When the externally expandable sub-implant 102 is assembled on the expansion aid 20, the interaction region 106 of the externally expandable sub-implant 102 constitutes the proximal end region 128 thereof, the proximal end region pointing toward the proximal end 44 of the insertion device 10. The seal structure 116 is additionally arranged at the interaction region 106. In the case of the self-expanding sub-implant 104, the interaction region 112 thereof is located in the state assembled in the protective sleeve 48 at a distal end region 118 of the self-expanding sub-implant 104, the distal end region pointing toward the distal end 34 of the insertion device 10. The interaction region 112 is thus arranged axially opposite the end region 120 with the fastening eyelets 126.

As can be seen from FIG. 1, each of the insertion elements 12, 14, 16, 18 and the stabilizing tube 40 has a grip segment 24, 26, 28, 30, 50, which are each designed to move the respective insertion element 12, 14, 16, 18 and the stabilizing tube 40. In this case, the grip segments 26, 28, 30, 50 of the insertion elements 14, 16, 18 and of the stabilizing tube 40 are each designed as hand grips extending substantially perpendicularly relative to a longitudinal axis of the proximal portion of the insertion device 10. In addition, the grip segments 26, 28, 30, 50 are each equipped with a Luer-Lock to remove air from the respective insertion element 14, 16, 18 or the stabilizing tube 40 (not shown). The grip segment 26 is connected separably to the insertion element 16 or to the grip segment 28, and the grip segment 28 is connected separably to the insertion element 12 (not illustrated in detail). When inserting the insertion device 10, a relative movement between the grip segment 26 and the insertion element 16 as well as between the grip segment 28 and the insertion element 12 can thus be prevented. The grip segment 50 is used to fix the stabilizing tube 40.

By contrast, the grip segment 24 of the first insertion element 12 is formed by a diverter 52 for inflation or deflation of the expansion aid 20 (see below). The grip segments 24, 30 are inseparably interconnected, whereby the insertion elements 12, 18 can be moved together both with the grip segment 24 and the grip segment 30. All grip segments 24, 26, 28, 30, 50 are each arranged at a proximal end 54-12, 54-14, 54-16, 54-18, 54-40 of the respective insertion element 12, 14, 16, 18 and of the stabilizing tube 40. In addition, the proximal shaft regions of the insertion elements 12, 14, 16, 18 can also be used as a grip segment.

The at least third insertion element 16 additionally has a stop 32, which limits a relative movement of the second insertion element 14 with respect to the third insertion element 16. This stop 32 is formed for example by a marking.

In the assembled state of the hybrid implant 100 in/on the insertion device 10, as can be seen in FIGS. 1 and 5, the first externally expandable sub-implant 102 faces the distal end 34 of the insertion device 10, and the second self-expanding sub-implant 104 faces away from the distal end 34 of the insertion device 10. In other words, the externally expandable sub-implant 102 is arranged distally from the self-expanding sub-implant 104. The interactions regions 106, 112 thus point toward one another.

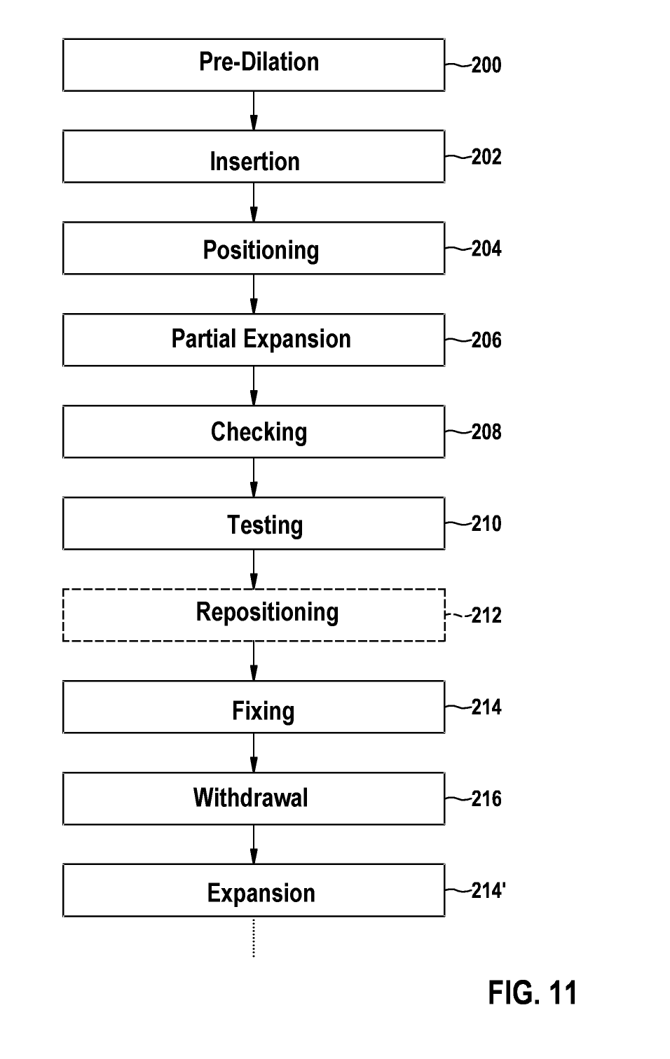

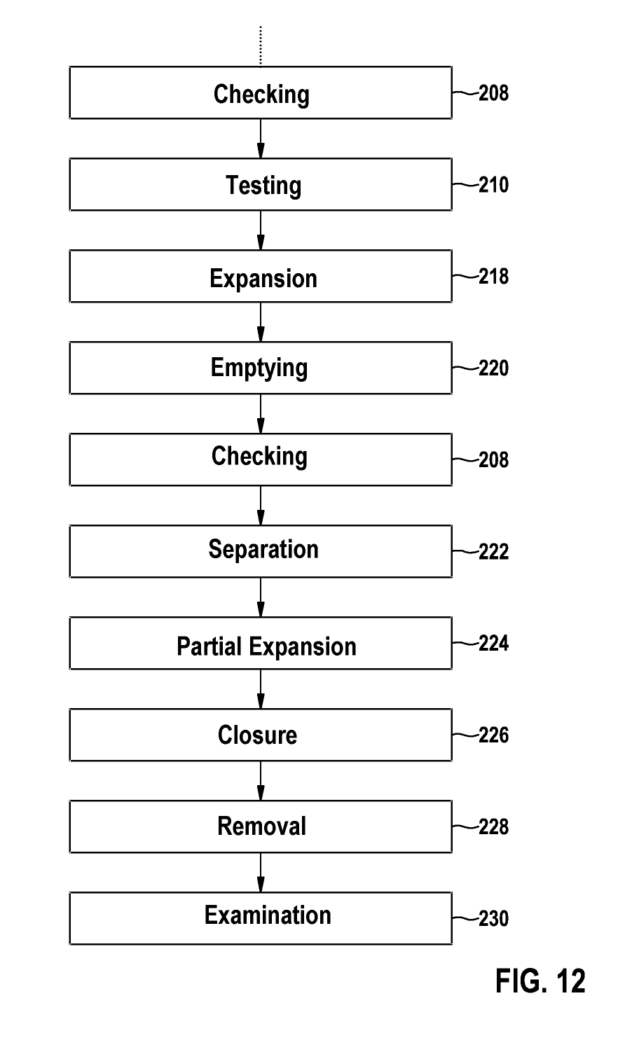

A method for inserting a medical hybrid implant 100 with the aid of the insertion device 10 is described hereinafter on the basis of FIGS. 5 to 10 and the block diagrams in FIGS. 11 and 12. The two sub-implants 102, 104 are assembled in/on the insertion device 10 (see FIG. 5), as described above and as is shown in FIG. 5.

Before the insertion device 10 is inserted, the native aortic valve is pre-dilated in step 200 (pre-dilation) using what is known as a valvuloplasty balloon. If the guide element 22 is not yet placed in the insertion device 10, this is then slid over the guide element 22 in step 202 (insertion). The insertion device 10 thus prepared is then inserted into the body in a known manner for implantation of the hybrid implant 100 in the body and is positioned in step 204 (positioning). In this case, a valve plane 110 of the valve insert 108 should be arranged flush with the annulus of the natural valve (not shown).

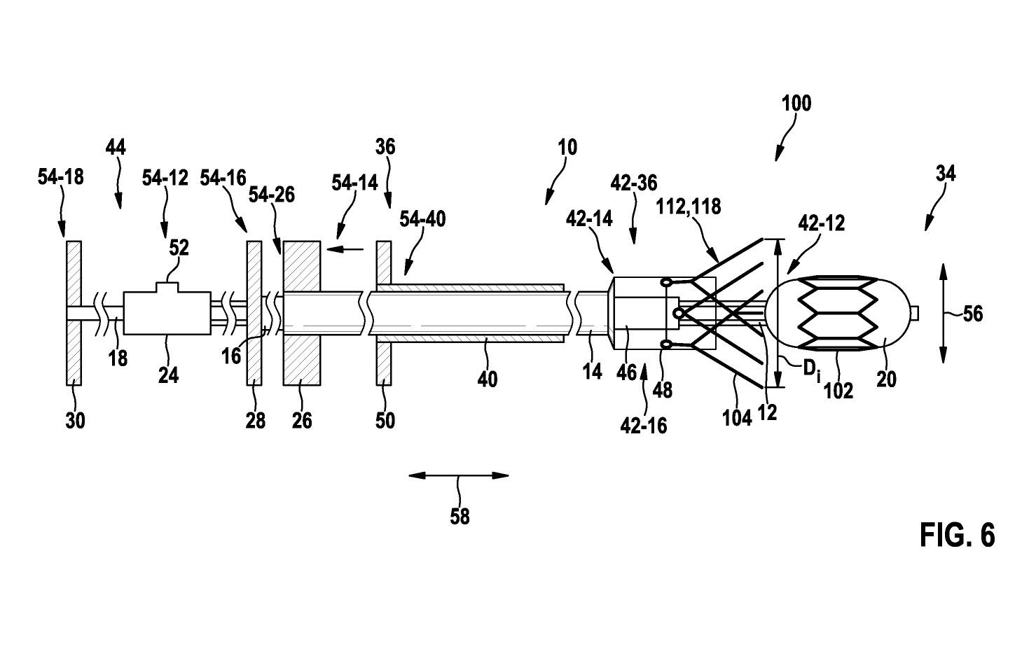

The hybrid implant 100 then starts to be placed in position. Here, a first sub-region 118 or the distal end region 118 of the self-expanding sub-implant 104 is exposed in a first step 206 (partial expansion), whereby the sub-implant expands automatically (see FIG. 6). In this case, the second insertion element 14 is drawn via the grip segment 26 in the direction of the proximal end 44 of the insertion device 10 (see arrow) once the connection between the grip segment 26 and the insertion element 16 or to the grip segment 28 has been separated, whereby the second insertion element 14 performs a relative movement in the axial direction 58 with respect to the other three insertion elements 12, 16, 18. The partial expansion of the first sub-region 118 is thus triggered by a relative movement of the second insertion element 14 with respect to the third insertion element 16. The drawing movement is carried out in this case until a proximal end 54-26 of the grip segment 26 of the second insertion element 14 is located at an axial height of the stop 32 of the third insertion element 16 (see the axial heights of the grip segment 26 in FIGS. 5 and 6). The relative movement is thus limited on account of the stop 32 (see FIG. 6).

The self-expanding sub-implant 104 is thus partly exposed and the interaction region 112, in its state thus expanded, has assumed an inner diameter Dᵢ, of which the dimension is adapted to a dimension of an outer diameter Dₐ of the interaction region 106 of the first externally expandable sub-implant 102 in the expanded state thereof. In this case, the inner diameter Dᵢ of the self-expanding sub-implant 104 is minimally larger than the outer diameter Dₐ of the externally expandable sub-implant 102, and the externally expandable sub-implant can therefore enter the inner diameter Dᵢ of the self-expanding sub-implant 104 (see below).

At this point, a position check of the expanded sub-region 118 can be carried out in step 208 (checking). In addition, a functional test of the hybrid implant 100 or of the valve insert 108 can then be carried out in step 210 (test). If an incorrect position of the first sub-region 118 is determined or if the valve insert 108 functions unsatisfactorily, the implant can then be repositioned in step 212 (repositioning). In this case, a resheathing process is carried out first. Here, the second insertion element 14 is again slid over the first sub-region 118 of the second self-expanding sub-implant 104 as a result of a relative movement in the axial direction 58 with respect to the third insertion element 16, whereby the sub-implant is pushed back into its starting position. The second insertion element 14 moves in the direction of the distal end 34 of the insertion device 10. The first sub-region 118 can then be position once more by again withdrawing the second insertion element 14. If malfunctioning is determined, the hybrid implant 100 can be removed again from the body. Since step 112 is an alternative method step, it is shown in FIG. 11 as a box with a dashed outline. If a correct position was reached, the second insertion element 14 is fixed proximally in step 214 (fixing) or is refixed via the grip segment 26 to the third insertion element 16 or to the grip segment 28.

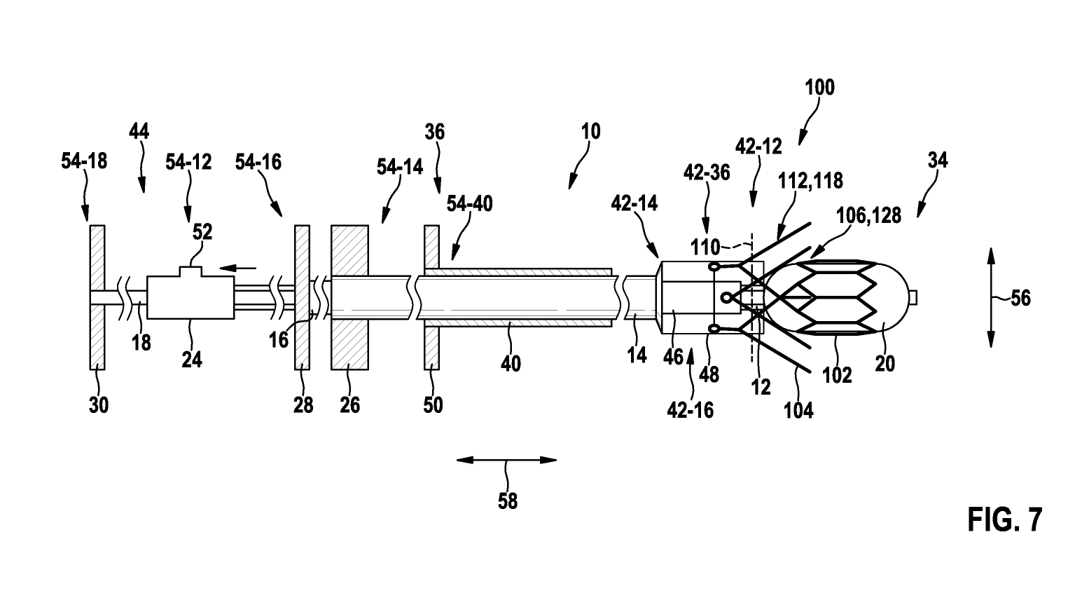

In a subsequent step 216 (withdrawal), the externally expandable sub-implant 102 and the expansion aid 20 are then positioned. Here, the interaction region 106 of the externally expandable sub-implant 102 enters the inner diameter Dᵢ of the interaction region 112 of the self-expanding sub-implant 104. However, this only occurs until the interaction region 106 is arranged distally from the valve plane 110 of the valve insert 108, thus avoiding damage to the valve insert 108 (see FIG. 7, in which the valve plane 110 is indicated merely schematically by a dashed line). The withdrawal takes place once the connection between the grip segment 28 and the insertion element 12 has been separated by moving the first insertion element 12 by pulling on the grip segment 30 together with the fourth insertion element 18 in the direction of the proximal end 44 of the insertion device 10 (see arrow). Alternatively, the first and the fourth insertion element 12, 18 may also be moved by pulling on the grip segment 24 or the diverter 52. As a result of the movement, the first insertion element 12 and the fourth insertion element 18 perform a relative movement in the axial direction 58 with respect to the second and third insertion elements 14, 16. The first insertion element 12 is thus arranged displacably inside the second insertion element 14. In reality, an axial spacing between the grip segment 26 and the grip segment 28 is larger than illustrated here, and therefore a movement of the grip segment 26 in the direction of the proximal end 44 of the insertion device 10 for complete release of the second sub-implant 104 is also possible (see below).

If the first externally expandable sub-implant 102 is then located in the desired and correct position, the first insertion element 12 is fixed proximally in step 114′ (fixing). As a precaution, the correct positions of the expanded distal sub-region 118 of the self-expanding sub-implant 104 and those of the externally expandable sub-implant 102 are examined again in step 208 (checking). Furthermore, the function of the valve insert 108 should be checked again here in step 210 (testing).

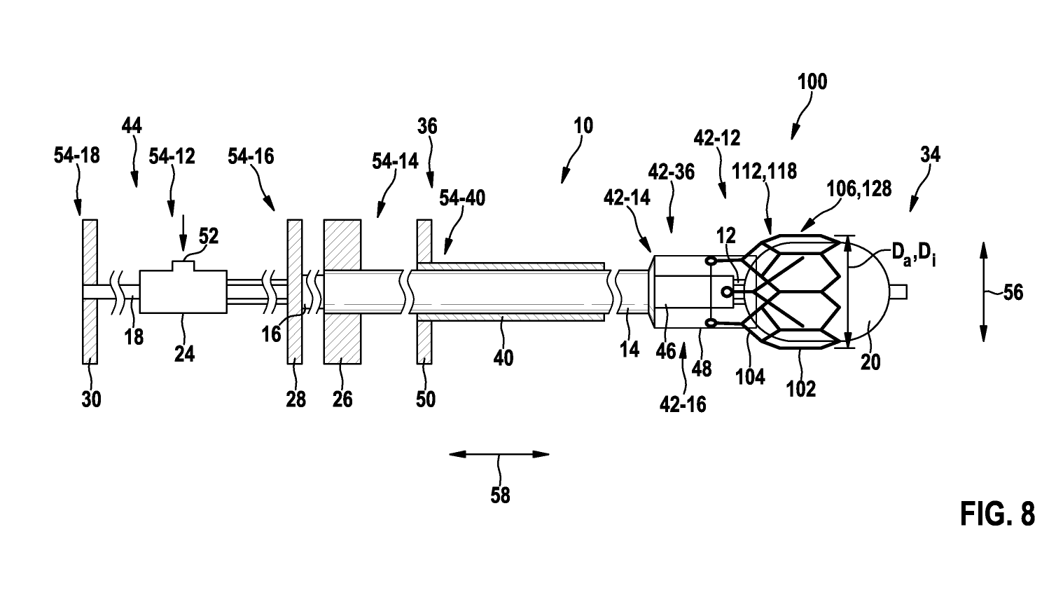

In a step 218 (expansion), the complete first externally expandable sub-implant 102 is then released (see FIG. 8). In this case, the expansion is carried out with the aid of the expansion aid 20. For this purpose, a medium (not illustrated in greater detail), such as compressed air or saline solution, is fed via the diverter 52 (see arrow), wherein the first insertion element 12 has a feed channel (not shown in greater detail) for this purpose. As already mentioned above, the interaction region 106 of the externally expandable sub-implant 102 has then assumed in the expanded state an outer diameter Dₐ, which is matched to the inner diameter Dᵢ of the interaction region 112 of the self-expanding sub-implant 104 (for reasons of improved presentability, the same extension is shown for both diameters, although the outer diameter Dₐ is minimally larger than the inner diameter Dᵢ). As a result of the expansion of the externally expandable sub-implant 102, the first sub-region 118 of the second self-expanding sub-implant 104 is thus fastened by means of the interaction region 106 of the first externally expandable sub-implant 102 at a destination site or the site of implantation (not shown in detail).

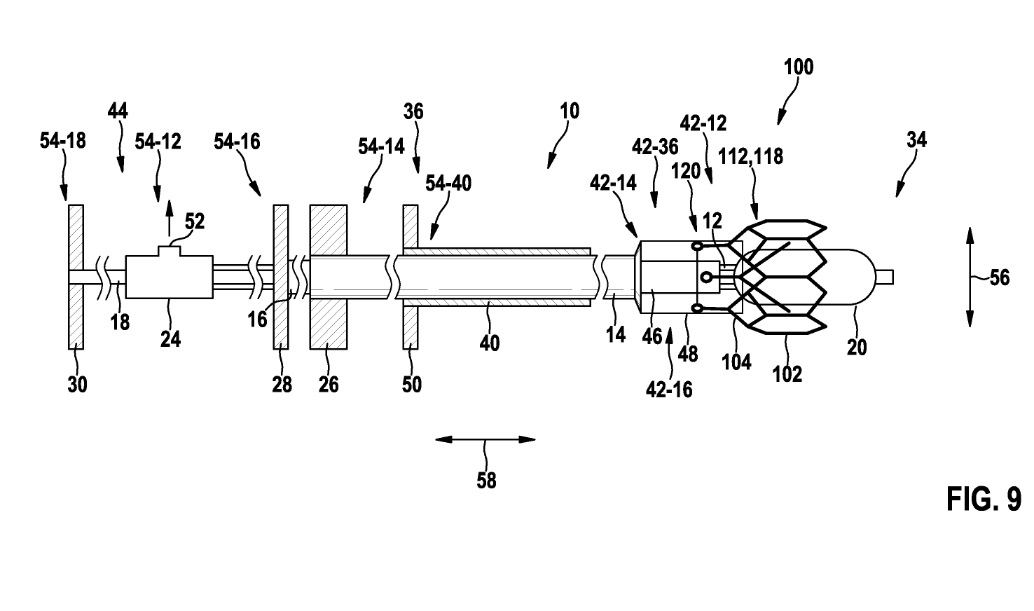

In the next step 220 (emptying), the medium is then removed from the expansion aid 20 (see arrow), whereby the expansion aid deflates. The externally expandable sub-implant 102 and therefore also the sub-region 118 of the self-expanding sub-implant 104 remain in their expanded state at the site of implantation (see FIG. 9). In step 208 (checking), the correct position of the expanded distal sub-region 118 of the self-expanding sub-implant 104 and that of the externally expandable sub-implant 102 is again examined.

Then, in step 222 (separation), the self-expanding sub-implant 104 is separated from the implant holder 46. This occurs for example by radially withdrawing the hooks of the implant holder 46 from the fastening eyelets 126 of the proximal end region 120 of the self-expanding sub-implant 104 (not shown in detail).

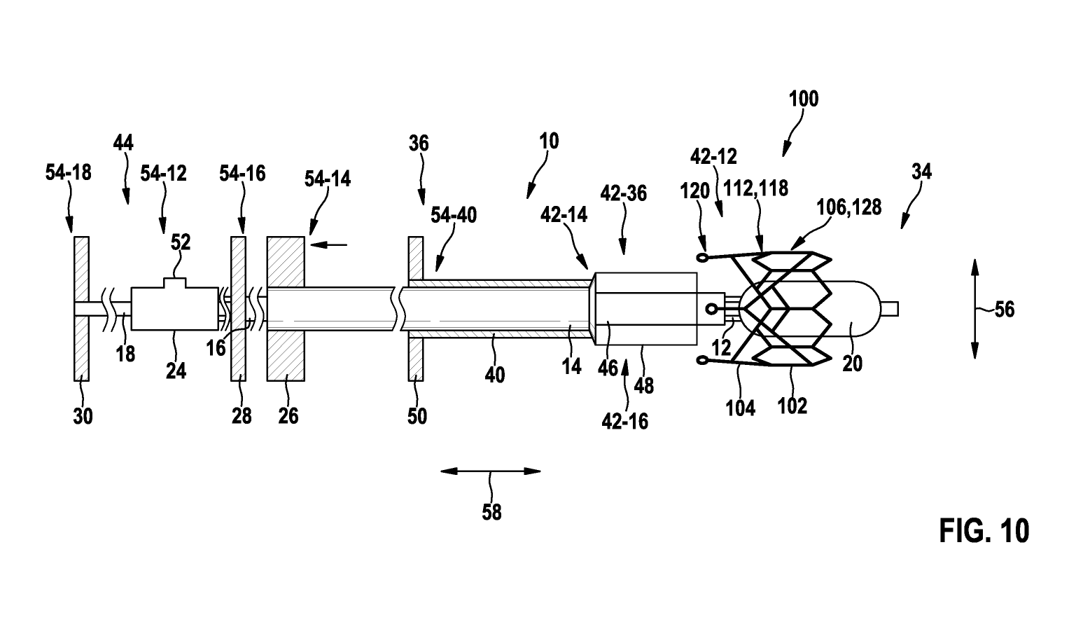

In the next step 224 (partial expansion), a second sub-region 120 or the proximal end region 120 of the second self-expanding sub-implant 104 is released, whereby the sub-implant expands automatically (see FIG. 10). In this case, the second insertion element 14 with the grip segment 26 is again drawn in the direction of the proximal end 44 of the insertion device 10 (see arrow) after separation of the fixing, whereby the second insertion element 14 performs a relative movement in the axial direction 58 with respect to the other three insertion elements 12, 16, 18. The partial expansion of the second sub-region 120 is thus triggered by a relative movement of the second insertion element 14 with respect to the third insertion element 16. In this case, the drawing movement occurs until the hybrid implant 100 is completely released.

Then, in step 226 (closure), the protective sleeve 48 is closed and the insertion device 10 is then withdrawn and removed from the body in step 228 (removal). The hybrid implant 100 remains fully positioned in the body (not shown). In order to verify the successful implantation, an aortogram is lastly taken in step 230 (examination).

In principle, one of the component parts/components of the insertion device 10 or of the hybrid implant 100 may also be formed from a metal that is visible under X-ray, such as stainless steel, tantalum, gold or platinum. An advance for example of the catheter tip 38 or of one of the insertion elements 12, 14, 16, 18 and therefore of the hybrid implant 100 as well as a correct position of the implant 102 at the site of implantation 10 as well as a correct position of the hybrid implant 100 at a site of implantation could thus be monitored with the aid of an X-ray device (not shown here) during the implantation of the hybrid implant 100 by means of the insertion device 10.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE SIGNS

10 insertion device
12 insertion element
14 insertion element
16 insertion element
18 insertion element
20 expansion aid
22 guide element
24 grip segment
26 grip segment
28 grip segment
30 grip segment
32 stop
34 end
36 shaft region
38 catheter tip
40 stabilizing tube
42 end
44 end
46 implant holder
48 protective sleeve
50 grip segment
52 diverter
54 end
56 direction
58 direction
100 hybrid implant
102 sub-implant
104 sub-implant
106 interaction region
108 valve insert
110 valve plane
112 interaction region
114 cell
116 seal structure
118 region
120 region
122 main framework
124 circumferential direction
126 fastening eyelets
128 end region
200 pre-dilation
202 insertion
204 positioning
206 partial expansion
208 checking
210 testing
212 repositioning
214 fixing
216 withdrawal
218 expansion
220 emptying
222 separation
224 partial expansion
226 closure
228 removal
230 examination
Dₐ outer diameter
Dᵢ inner diameter

What is claimed is:

1. An insertion device for inserting a medical hybrid implant, wherein the hybrid implant comprises at least two sub-implants, of which at least one first sub-implant is externally expandable and at least one second sub-implant is self-expanding, the insertion device comprising a first insertion element, which comprises at least one expansion aid for expanding the at least first externally expandable sub-implant, and a second insertion element and at least one third insertion element for releasing the at least second self-expanding sub-implant, wherein the at least first externally expandable sub-implant is expandable by means of the expansion aid and the at least second self-expanding sub-implant can be released by a relative movement between the second insertion element and the at least third insertion element, wherein the first insertion element is arranged displacably inside the at least second insertion element.

2. The insertion device as claimed in claim 1, wherein the at least third insertion element is arranged radially between the first insertion element and the second insertion element and/or is configured to hold the second self-expanding sub-implant in position, at least in the event of an expansion of the second self-expanding sub-implant.

3. The insertion device as claimed in claim 1, further comprising at least one fourth insertion element, which is arranged radially inside the first insertion element and/or which is configured to receive a guide element.

4. The insertion device as claimed in claim 1, further comprising at least one fourth insertion element, and/or wherein the second insertion element is arranged axially displacably with respect to the at least first insertion element and/or with respect to the at least fourth insertion element.

5. The insertion device as claimed in claim 4, wherein the first insertion element and/or the second insertion element and/or the third insertion element and/or the at least fourth insertion element each has at least one grip segment, and/or wherein the grip segment is in each case designed, in at least one operating state, to move the first and the at least fourth insertion element independently of the second and the at least third insertion element, and/or wherein the grip segment is designed, in at least one operating state, to move the second insertion element independently of each of the first, the at least third, and the at least fourth insertion element.

6. The insertion device at least as claimed in claim 4, wherein the at least third insertion element has a stop, which limits a relative movement of the second insertion element with respect to the at least third insertion element.

7. The insertion device as claimed in claim 1, wherein, in an assembled state of the hybrid implant, the at least first externally expandable sub-implant faces a distal end of the insertion device and/or the at least second self-expanding sub-implant faces away from the distal end of the insertion device.

8. The insertion device as claimed in claim 1, wherein the first externally expandable sub-implant has at least one interaction region for interaction with the at least second self-expanding sub-implant, and the at least second self-expanding sub-implant has at least one valve insert, wherein the at least one interaction region of the first externally expandable sub-implant can be arranged distally from a valve plane of the valve insert as a result of a relative movement between the first insertion element and the second insertion element of the insertion device.

9. A medical hybrid implant, in particular to be inserted by means of an insertion device as claimed at least in claim 1, comprising at least two sub-implants, of which at least one first sub-implant is externally expandable and at least one second sub-implant is self-expanding, wherein at least one interaction region of the first externally expandable sub-implant has an outer diameter (Dₐ) in the expanded state and at least one interaction region of the at least second self-expanding sub-implant has an inner diameter (Dᵢ) in the expanded state, wherein a dimension of the outer diameter (Dₐ) of the at least one interaction region of the first externally expandable sub-implant is adapted to a dimension of the inner diameter (Dᵢ) of the at least one interaction region of the at least second self-expanding sub-implant, so that, in the event of a relative movement between a first insertion element and a second insertion element of the insertion device, at least the one interaction region of the first externally expandable sub-implant can enter the at least one interaction region of the at least second self-expanding sub-implant, and the interaction region of the second self-expanding sub-implant can be fastened in the expanded state at a destination site by means of the interaction region of the first externally expandable sub-implant.

10. The medical hybrid implant as claimed in claim 9, wherein the at least second self-expanding sub-implant has at least one valve insert.

11. The medical hybrid implant as claimed in claim 9, wherein the at least one interaction region of the first externally expandable sub-implant can be arranged distally from a valve plane of the valve insert as a result of the relative movement between the first insertion element and the second insertion element of the insertion device.

12. The medical hybrid implant as claimed in claim 9, wherein the at least second self-expanding sub-implant has a number of cells, wherein the number is selected such that the second self-expanding sub-implant has a low radial force.

13. The medical hybrid implant as claimed in claim 9, wherein the at least first externally expandable sub-implant has at least one seal structure, which is designed, in the implanted state, to reduce a leak.

14. A method for inserting a medical hybrid implant with the aid of an insertion device as claimed in claim 1, wherein the medical hybrid implant comprises at least two sub-implants, of which at least one first sub-implant is externally expandable and at least one second sub-implant is self-expanding, wherein at least one interaction region of the first externally expandable sub-implant has an outer diameter (Dₐ) in the expanded state and at least one interaction region of the at least second self-expanding sub-implant has an inner diameter (Dᵢ) in the expanded state, wherein a dimension of the outer diameter (Dₐ) of the at least one interaction region of the first externally expandable sub-implant is adapted to a dimension of the inner diameter (Dᵢ) of the at least one interaction region of the at least second self-expanding sub-implant, so that, in the event of a relative movement between a first insertion element and a second insertion element of the insertion device, at least the one interaction region of the first externally expandable sub-implant can enter the at least one interaction region of the at least second self-expanding sub-implant, and the interaction region of the second self-expanding sub-implant can be fastened in the expanded state at a destination site by means of the interaction region of the first externally expandable sub-implant; the method comprising at least the following steps:
a) partially expanding a first sub-region of the at least second self-expanding sub-implant, in particular of a distal end region of the at least second self-expanding sub-implant when the at least second self-expanding sub-implant is assembled on the insertion device;
b) expanding a complete at least first externally expandable sub-implant; and
c) partially expanding a second sub-region of the at least second self-expanding sub-implant, in particular of a proximal end region of the at least second self-expanding sub-implant when the at least second self-expanding sub-implant is assembled on the insertion device.

15. The method as claimed in claim 14, wherein,
the partial expansion of the first sub-region of the at least second self-expanding sub-implant is triggered by a relative movement of a second insertion element with respect to a third insertion element of the insertion device, wherein the relative movement is limited on account of a stop of an at least third insertion element;
the expansion of the first externally expandable sub-implant is carried out with the aid of at least one expansion aid, whereby the first sub-region of the second self-expanding sub-implant is fastened at a destination site by means of an interaction region of the first externally expandable sub-implant; and
the partial expansion of the second sub-region of the at least second self-expanding sub-implant is triggered by a relative movement of the second insertion element with respect to the third insertion element of the insertion device, whereby the hybrid implant is completely released.
